(19) United States
(12) Patent Application Publication
(10) Pub. No.: US 2023/0303965 A1
(43) Pub. Date: Sep. 28, 2023

(54) NOVEL LACTOBACILLUS REUTERI STRAIN AND USE THEREOF

(71) Applicant: KOREA RESEARCH INSTITUTE OF BIOSCIENCE AND BIOTECHNOLOGY, Daejeon (KR)

(72) Inventors: Mi-Young SON, Daejeon (KR); Doo-Sang PARK, Daejeon (KR); Kwang Bo JUNG, Daejeon (KR); Hana LEE, Daejeon (KR)

(73) Assignee: KOREA RESEARCH INSTITUTE OF BIOSCIENCE AND BIOTECHNOLOGY, Daejeon (KR)

(21) Appl. No.: 18/003,047

(22) PCT Filed: Jun. 23, 2021
(86) PCT No.: PCT/KR2021/007921

§ 371 Date: Dec. 22, 2022

(30) Foreign Application Priority Data
Jun. 23, 2020 (KR) 10-2020-0076569

(51) Int. Cl. C12N 1/20; A61K 35/747; A61P 1/00

(52) U.S. Cl. CPC C12N 1/205; A61K 35/747; A61P 1/00; C12R 2001/225

(57) ABSTRACT

The present invention relates to a novel Lactobacillus reuteri DS0384 strain and the use of thereof.

### CROSS-REFERENCE TO RELATED APPLICATIONS

This application is a national phase under 35 U.S.C. § 371 of PCT International Application No. PCT/KR2021/007921 which has an International filing date of Jun. 23, 2021, which claims priority to Korean Application No. 10-2020-0076569, filed Jun. 23, 2020,the entire contents of each of which are hereby incorporated by reference.

### SEQUENCE LISTING

This application is being filed electronically via the USPTO EFS-WEB server, as authorized and set forth in MPEP §502.5 and this electronic filing includes an electronically submitted sequence listing. The entire content of this sequence listing is hereby incorporated by reference into the specification of this application. The sequence listing is identified on the electronically filed ASII(.txt) text file as follows:

| | | |
|---|---|---|
| File Name | Date of Creation | Size |
| 17597KI-000010-US-NP_SequenceListing.txt | Feb. 21, 2023 | 4.00 KB |

### TECHNICAL FIELD

The present invention relates to a novel Lactobacillus Reuteri strain and use of thereof.

### BACKGROUND ART

The “intestine,” which consists of the small intestine and large intestine, is an organ that plays a key role in food digestion by secreting various kinds of digestive enzymes, and is also an organ that plays an important role in absorbing digested nutrients, water, and the like, and secretes hormones. Food that is ingested through the mouth to undergo digestion process is absorbed into the body as it travels along the intestinal tract, wherein nutrients including amino acids and glucose are absorbed from the villi of the small intestine, and most of the water is absorbed from the large intestine. The development of structures, such as the length and area of the intestinal tract, which is a passageway through which food passes and provides a cross-section through which digested food comes into contact, and the villi of the intestine, allow for normal digestion and absorption.

In addition to digestive and absorptive functions, the intestine is an organ closely related to the microbiome, and about 95% of the microorganisms present in the human body inhabit the intestine, and the number of microorganisms more than 10 times the number of cells in the human body is present in the human intestine. As research results reveal that the types of intestinal microorganisms, the number and ratio of each type of microorganisms, and the like may have an important effect on human health or physical characteristics, interest in the correlation between intestinal microorganisms and human diseases, health, and the like is increasing.

Thus, if the length of the intestine is short, the cross-sectional area is small, or the development of the detailed structure of the intestine is insufficient, compared with a normal person, due to problems with the development or maturation of the intestine, there is a possibility that the digestive and absorptive capacities decrease, which adversely affects the overall health. In particular, if intestinal development is slow in fetuses, neonates, infants, and the like who have not completed development and growth, it also adversely affects the development or overall growth of organs other than the intestine, and thus, the need for improvement thereof is higher, and even if there are no disorders in intestinal development, it can be said that the importance of research on methods capable of helping the intestinal development and maturation in developing and growing individuals is high.

On the other hand, Lactobacillus reuteri is a kind of lactic acid bacteria that inhabit the intestine, and is known to have various functions. Among the published prior patent documents, there are a report that a Lactobacillus reuteri strain has an effect of preventing and improving skin aging (Korean Laid-open Patent Publication No. 10-2020-0028627), a report on a Lactobacillus reuteri strain having an immune enhancing effect (Korean Laid-open Patent Publication No. 10-2018-0053499), and a report on a strain enhancing the development of a “nervous system” such as intestinal neuronal cells (Korea Laid-open Patent Publication No. 10-2014-0131501), but prior patents disclosing a Lactobacillus reuteri strain exhibiting the property of promoting intestinal development and maturation have not been reported. In particular, there have been no studies on strains that are particularly superior to other strains in the effect of promoting the expression of genes and proteins specifically expressed in the mature intestinal tract, or studies on their applicability to the human intestine, and thus, there is a need for development of a novel Lactobacillus reuteri strain that is excellent in the intestinal development and maturation effects and also has the characteristics applicable to human intestinal models.

### DISCLOSURE

### Technical Problem

It is an object of the present invention to provide a novel lactic acid bacteria strain that is superior compared to other microbial strains in the effect of promoting intestinal development or intestinal maturation and has the characteristics capable of exhibiting the effect of protecting and recovering the damaged intestine.

In addition, it is another object of the present invention to provide a fermentation starter composition for use in the production of fermented food or the like or for use in the production of a strain culture broth, using the novel lactic acid bacteria strain as described above.

In addition, it is another object of the present invention to provide a composition that may be used for the purpose of promoting intestinal development or maturation or recovery of intestinal damage using the novel lactic acid bacteria strain as described above.

In addition, it is another object of the present invention to provide a pharmaceutical composition that may be used for the purpose of preventing or treating intestinal development disorders or inflammatory bowel disease using the novel lactic acid bacteria strain as described above.

In addition, it is another object of the present invention to provide a health functional food composition and a feed composition, that may be used for the purpose of preventing or improving intestinal development disorders or inflammatory bowel disease using the novel lactic acid bacteria strain as described above.

### Technical Solution

In order to achieve the above objects, an aspect of the present invention provides a Lactobacillus reuteri DS0384 strain.

In order to achieve the above objects, another aspect of the present invention provides a fermentation starter composition comprising a Lactobacillus reuteri DS0384 strain.

In order to achieve the above objects, another aspect of the present invention provides a composition for promoting intestinal development, intestinal maturation or intestinal damage recovery, comprising a Lactobacillus reuteri DS0384 strain or its culture broth.

In order to achieve the above objects, another aspect of the present invention provides a pharmaceutical composition for preventing or treating intestinal development disorders or inflammatory bowel disease, comprising a Lactobacillus reuteri DS0384 strain or its culture broth.

In order to achieve the above objects, another aspect of the present invention provides a health functional food composition and a feed composition for preventing or improving intestinal development disorders or inflammatory bowel disease, comprising a Lactobacillus reuteri DS0384 strain or its culture broth.

### Advantageous Effects

The novel Lactobacillus reuteri DS0384 strain of the present invention and its culture broth have the effects of increasing the size of intestinal organoids made by differentiation from human intestinal stem cells, increasing the budding structure, and increasing the expression of mature intestinal marker genes and proteins.

In addition, even when the human intestinal stem cells isolated from the intestinal organoids are treated with the Lactobacillus reuteri DS0384 strain and its culture broth, there is the effect of promoting the growth of intestinal stem cells.

In addition, when intestinal organoids is simultaneously treated with the Lactobacillus reuteri DS0384 strain together with inflammatory cytokines, the surface area of the damaged intestine may increase and the expression level of the intestinal wall function and proliferation-related marker proteins may increase, and thus, there is also excellent in the effect of promoting and improving recovery of intestinal damage.

Furthermore, even when mice are orally gavaged with the Lactobacillus reuteri DS0384 strain of the present invention and its culture broth, intestinal development is promoted, such as the length and area of the villus and the depth of the crypt in the small intestine increase and the mucosa/submucosa ratio of the large intestine increases, and the expression of mature intestinal marker genes and proteins increases.

In particular, the Lactobacillus reuteri DS0384 strain of the present invention exhibits the effects described above more remarkably even when compared to lactic acid bacteria belonging to other species as well as other strains classified as Lactobacillus reuteri, and has the characteristic of increasing the expression of mature intestinal marker genes, which cannot be increased in other Lactobacillus reuteri strains, and thus, it corresponds to a novel strain having characteristics and effects that are not found and so cannot be predicted in conventional strains. In addition, it was confirmed that when treated with the DS0384 strain, the effects were not only seen in non-human animals, but also in intestinal organoids and human intestinal stem cells prepared from a human intestinal model, and thus, it could be newly confirmed that the excellent effect of promoting intestinal development and intestinal maturation and promoting and improving recovery of intestinal damage was also shown in humans.

Therefore, the Lactobacillus reuteri DS0384 strain of the present invention may be used for the purpose of promoting intestinal development or intestinal maturation, or usefully used as drugs, foods and feeds for preventing, treating and improving intestinal development disorders, and thus is very useful in related industries.

### BRIEF DESCRIPTION OF DRAWINGS

FIG. 1A shows the results of confirming the morphological changes of intestinal organoids under a microscope (upper data; bright field, BF) and the results of comparing the expression of mature intestinal marker proteins through immunofluorescence staining (lower data), after treating the intestinal organoids with the culture broth of B. longum, L. gasseri, L. curvatus, L. rhamnosus, and the Lactobacillus reuteri (L. reuteri) DS0384 strain of the present invention. On the scale bar, black indicates 500 µm and white indicates 100 µm. FIG. 1B shows a graph comparing the size changes of intestinal organoids (left panel) and the number of budding structures of the intestinal organoids (right panel) to confirm the morphological changes of the intestinal organoids, after treating the intestinal organoids with the culture broth of B. longum, L. gasseri, L. curvatus, L. rhamnosus, and the Lactobacillus reuteri (L. reuteri) DS0384 strain of the present invention. (*: p<0.05 according to t-test, control group vs experimental group; **: p<0.01 according to t-test, control group vs experimental group; ***: p<0.001 according to t-test, control group vs experimental group) FIG. 1C shows a graph comparing the expression level of mature intestinal marker genes (CDX2, DPP4, OLFM4, DEFA5, CREB3L3, KRT20, LYZ, LCT, SLC5A1, and MUC13) in the intestinal organoids treated with the Lactobacillus reuteri (L. reuteri) DS0384 strain of the present invention, confirmed by qRT-PCR, and the result of the control group. (*: p<0.05 according to t-test, control group vs experimental group; **: p<0.01 according to t-test, control group vs experimental group)

FIG. 2A shows the results of confirming the morphological changes of intestinal organoids under a microscope (upper data; bright field, BF) and the results of comparing the expression of mature intestinal marker proteins through immunofluorescence staining (lower data), after treating the intestinal organoids with the culture broth of DS0191, DS0195, DS0333 and DS0354 classified as Lactobacillus reuteri and the Lactobacillus reuteri DS0384 strain of the present invention. On the scale bar, black indicates 500 µm and white indicates 100 µm. FIG. 2B shows a graph comparing the expression level of mature intestinal marker genes (CDX2, DPP4, OLFM4, DEFA5, CREB3L3, KRT20, LYZ, LCT, SLC5A1, and MUC13), confirmed by qRT-PCR. (*: p<0.05 according to t-test, control group vs experimental group; **: p<0.01 according to t-test, control group vs experimental group)

FIG. 3A shows the results of confirming the morphological changes of intestinal organoids under a microscope (upper data; bright field, BF) and the results of comparing the expression of mature intestinal marker proteins through immunofluorescence staining (lower data), after treating the intestinal organoids with the culture broth of DSP007, DS0337 and KCTC3594 classified as Lactobacillus reuteri and the Lactobacillus reuteri DS0384 strain of the present invention. On the scale bar, black indicates 500 µm and white indicates 100 µm. FIG. 3B shows a graph comparing the expression level of mature intestinal marker genes (CDX2, DPP4, OLFM4, DEFA5, CREB3L3, KRT20, LYZ, LCT, SLC5A1, and MUC13), confirmed by qRT-PCR. (*: p<0.05 according to t-test, control group vs experimental group; **: p<0.01 according to t-test, control group vs experimental group; ***: p<0.001 according to t-test, control group vs experimental group)

FIG. 4A shows the results of confirming the morphological changes of intestinal organoids, after treating the intestinal organoids with the culture broth obtained by culturing the Lactobacillus reuteri DS0384 strain and the DSP007 strain for 6 hours, 12 hours, 18 hours and 24 hours, respectively. Scale bar indicates 500 µm. FIG. 4B shows a graph comparing the expression patterns of the mature intestinal marker genes, which appear in the intestinal organoids treated with the culture broth of the Lactobacillus reuteri strains obtained for each time period, confirmed through qRT-PCR. (*: p<0.05 according to t-test, control group vs experimental group; **: p<0.01 according to t-test, control group vs experimental group)

FIG. 5A shows the results of confirming the morphological changes of human intestinal stem cells, after treating the intestinal stem cells isolated from intestinal organoids with the culture broth of the Lactobacillus reuteri DS0384 strain and the KCTC3594 strain. On the scale bar, black indicates 1 mm and white indicates 200 µm. FIG. 5B shows an analysis of the relative size of the surface area of intestinal stem cell colonies treated with the culture broth of the strains using the imageJ program. (n≥5 per group; *: p<0.05 according to t-test, control group vs experimental group; **: p<0.01 according to t-test, control group vs experimental group)

FIG. 6A is a drawing confirming the morphological changes of intestinal organoids in the control group in which the intestinal organoids are treated with the inflammatory cytokines IFNγ/TNFα for 3 days (control), and the group in which the intestinal organoids are treated with both the inflammatory cytokines and the culture broth of the Lactobacillus reuteri DS0384 strain (bright field, BF). Scale bar indicates 1 mm. FIG. 6B is a drawing confirming the morphological changes of intestinal organoids through hematoxylin and eosin staining in the control group treated with PBS (control), the control group treated only with the inflammatory cytokines (IFNγ/TNFα), and the group treated with both the inflammatory cytokines and the culture broth of the Lactobacillus reuteri DS0384 strain. Scale bar indicates 200 µm. FIG. 6C is a graph showing the numerical comparison of changes in the surface area of intestinal organoids in the control group and the group treated with the culture broth of the Lactobacillus reuteri DS0384 strain. (n≥10 per group; *: p<0.05 according to t-test, control group vs experimental group)

FIG. 7 shows a drawing confirming the expression of the intestinal wall function marker ZO-1 protein and the proliferation marker Ki67 protein of intestinal organoids through immunofluorescence staining in the control group in which the intestinal organoids are treated with PBS (control), the control group in which the intestinal organoids are treated with the inflammatory cytokines IFNγ/TNFα for 3 days (IFNγ/TNFα), and the group in which the intestinal organoids are treated with both the inflammatory cytokines and the culture broth of the Lactobacillus reuteri DS0384 strain. On the scale bar, white indicates 125 µm and yellow indicates 500 µm. The graph below compares the expression level of ZO-1 and Ki67 measured as described above. (n≥5 per group; *: p<0.05 according to t-test, control group vs experimental group; ***: p<0.001 according to t-test, control group vs experimental group)

FIG. 8 shows the results of confirming the intestinal tissue of mice orally gavaged with the culture broth containing bacteria in the culture broth of the Lactobacillus reuteri DS0384 strain of the present invention (“strain” in the figure) and the culture broth from which bacteria were removed by centrifugation (“culture broth” in the figure), and the intestinal tissue of mice orally gavaged with the culture broth of Lactobacillus reuteri KCTC3594 and PBS, through histological morphology analysis method after hematoxylin and eosin staining.

FIG. 9 is a graph showing the numerical comparison of changes in the length and area of the villus and the depth of the crypt in the jejunum of the small intestine, and the depth of the crypt and the mucosa/submucosa ratio in the large intestine (colon), in the results according to FIG. 8 above. (n>7 per group; *: p<0.05 according to t-test, control group vs experimental group; **: p<0.01 according to t-test, control group vs experimental group; ***: p<0.001 according to t-test, control group vs experimental group)

### BEST MODE

Hereinafter, the present invention will be described in detail.

In the present invention, the term “culture broth” refers to the culture broth itself obtained by culturing a strain, or the culture supernatant obtained by removing the strain therefrom, and a filtrate, concentrate or dry matter product thereof, and may be used interchangeably with “culture supernatant,” “conditioned culture broth,” or “conditioned medium.”

In the present invention, the term “prevention” refers to any action that suppresses or delays the onset of a corresponding disease or negative condition by administering a composition to a subject.

In the present invention, the term “treatment” may be any action that turns around the symptoms of a corresponding disease or negative condition that has already occurred by administering a composition to a subject.

In the present invention, the term “improvement” refers to any action including turnaround, suppression, or delay of symptoms, and may be used interchangeably with the prevention or treatment.

### 1. Novel Lactobacillus Reuteri Strain and a Fermentation Starter Composition Comprising the Same

An aspect of the present invention provides a novel Lactobacillus reuteri strain.

The Lactobacillus reuteri is Lactobacillus reuteri DS0384, and is a strain deposited under Accession No. KCTC 14164BP. The Lactobacillus reuteri DS0384 strain was deposited with the Korean Collection for Type Cultures, the Korea Research Institute of Bioscience and Biotechnology under Accession No. KCTC 14164BP as of Apr. 6, 2020.

The Lactobacillus reuteri DS0384 strain may be a strain capable of inhabiting the intestine of humans or non-human animals, and may have various effects on the health of humans or non-human animals while inhabiting the intestine. General microorganisms of the genus Lactobacillus are Gram-positive anaerobic bacteria, which can ferment to produce lactic acid, acetic acid and the like in the intestine, and is known as a lactic acid bacterium having effects, such as inhibiting the growth of harmful bacteria, or enhancing immunity, promoting digestion and improving intestinal diseases in humans or non-human animals in which the microorganisms inhabit. The Lactobacillus reuteri DS0384 strain is a microorganism classified into the genus Lactobacillus, and may exhibit some of the characteristics of the microorganisms of the genus Lactobacillus as described above. The Lactobacillus reuteri DS0384 strain may be a safe microorganism because it does not show toxicity or cause disease to humans or non-human animals, and may function as a probiotic microorganism because it can act as a beneficial bacterium that helps the health of humans or non-human animals in the intestine.

The Lactobacillus reuteri DS0384 strain may have the characteristic of promoting intestinal development or intestinal maturation. The Lactobacillus reuteri DS0384 strain may have the activity of promoting the small intestine or large intestine to become a more mature state, or may have the activity of promoting the maturation of the small intestine or large intestine in an immature state. For example, the strain may increase the length and area of the villus or increase the depth of the crypt in the small intestine, or increase the depth of the crypt or increase the mucosa/submucosa ratio in the large intestine. In addition, the strain may increase the expression of one or more genes selected from the group consisting of CDX2, DPP4, OLFM4, DEFA5, CREB3L3, KRT20, LYZ, LCT, SLC5A1, and MUC13.

In a specific embodiment of the present invention, when the intestinal organoids prepared from the exo vivo intestinal model of the human body were treated with the culture broth containing the metabolites produced by the strain as the Lactobacillus reuteri DS0384 strain is cultured, it was confirmed that the maturation and development were promoted and the expression level of maturation-related marker genes and proteins increased. In addition, it was confirmed that the surface area of the stem cell colonies increased compared to the control group even when the intestinal stem cells isolated from the intestinal organoids as well as the intestinal organoids were treated with the culture broth of the Lactobacillus reuteri DS0384 strain, and thus, it was also confirmed that there was the activity of promoting the growth and maturation of intestinal stem cells. The intestinal organoids are prepared by differentiating human pluripotent stem cells, and embody a human exo vivo intestinal model, and the intestinal stem cells isolated from the intestinal organoids are also human intestinal stem cells. Therefore, it could be newly confirmed that Lactobacillus reuteri is a strain having the excellent activity of promoting intestinal development or maturation in humans as well as non-human animals, and thus, it could be confirmed that it is a lactic acid bacteria species that can usefully used for the purpose of promoting human intestinal development or maturation.

In another specific embodiment of the present invention, it was confirmed that the development of the small intestine and large intestine was promoted and the expression of mature intestinal marker genes increased even when mice were orally gavaged with the culture broth containing bacteria of the Lactobacillus reuteri DS0384 strain or the culture broth containing the metabolites produced by the strain. In particular, it was confirmed that the Lactobacillus reuteri DS0384 strain exhibited the effect of promoting intestinal maturation that was not shown in lactic acid bacteria classified as other species, and showed remarkably higher the expression level of genes and proteins related to intestinal maturation, or increased the expression of mature intestinal-related marker genes, which were not increased in the groups treated with the culture broth of other microorganisms, even when compared to the case of treatment with the culture broth of 7 types of microorganisms classified as Lactobacillus reuteri. Therefore, the Lactobacillus reuteri DS0384 strain is a novel strain that exhibits the effect of promoting intestinal maturation and intestinal development at levels that cannot be predicted or achieved in conventionally known lactic acid bacteria or Lactobacillus reuteri microorganisms, and thus, the strain of the present invention may be effectively used for the purpose of promoting intestinal development or intestinal maturation, or preventing, treating, or improving intestinal development disorders.

The Lactobacillus reuteri DS0384 strain may have the characteristic of promoting recovery of intestinal damage. The intestinal damage may be, for example, caused by an inflammatory response in the intestine, but the cause of the intestinal damage is not limited thereto, and if the function or structure of the intestine is damaged compared to a normal state, the Lactobacillus reuteri strain of the present invention may promote its recovery.

The recovery of intestinal damage may be to restore the intestine to its original state by increasing the surface area of the damaged intestine, or to reduce damage by protecting the intestine from damage. In addition, the recovery of intestinal damage may be to increase the expression of gene or protein markers related to the intestinal wall function or proliferation in the damaged intestine. The intestinal wall function marker may be, but is not limited to, a ZO-1 protein, and the intestinal wall proliferation marker may be, but is not limited to, a Ki67 protein.

In a specific embodiment of the present invention, when intestinal organoids were simultaneously treated with the culture broth containing the metabolites produced by the Lactobacillus reuteri DS0384 strain together with inflammatory cytokines, it was confirmed that the surface area increased and the damaged surface area was restored, and it was also confirmed that the expression level of ZO-1 protein and Ki67 protein increased, and thus, it could be confirmed that the Lactobacillus reuteri DS0384 strain had the activity of preventing intestinal damage and the activity of promoting recovery of the damaged intestine.

Another aspect of the present invention provides a fermentation starter composition.

The fermentation starter composition comprises a Lactobacillus reuteri DS0384 strain.

The description of the Lactobacillus reuteri DS0384 strain is the same as described above.

In the present invention, the term “fermentation starter” refers to a preparation comprising a microorganism involved in fermentation and other ingredients that provide essential ingredients for the growth of the microorganism, and is used for producing the fermented products or metabolites by the microorganism in large quantities or culturing microorganism involved in the fermentation in large quantities. In the fermentation starter composition of the present invention, the microorganism involved in the fermentation comprises a Lactobacillus reuteri DS0384 strain. The fermentation starter composition may comprise the culture broth of the Lactobacillus reuteri DS0384 strain.

The fermentation starter composition may be a stock culture or a seed culture that were conservatively cultured to preserve and store the Lactobacillus reuteri DS0384 strain for a long period of time, and may be a mother starter produced from the stock culture or seed culture, or may be a bulk starter produced using the mother starter. The fermentation starter composition may be used in the manufacture of a composition for promoting intestinal development or intestinal maturation comprising a Lactobacillus reuteri DS0384 strain or its culture broth, a pharmaceutical composition for preventing or treating intestinal development disorders, a health functional food composition for preventing or improving intestinal development disorders, a feed composition for preventing or improving intestinal development disorders, a fermented food, and the like, and the description of the composition for promoting intestinal development or intestinal maturation, the pharmaceutical composition, the health functional food composition and the feed composition is the same as described below.

The fermented food may be, but is not limited to, yogurt (hard type, soft type, drink type), fermented milk such as lactic acid bacteria beverage, cheese, or butter, and any food or product manufactured by fermentation performed by fermenting microorganisms or lactic acid bacteria may also be included.

The fermentation starter composition of the present invention may be used in any preparation, product, food, and the like manufactured for the purpose of using the effect of the Lactobacillus reuteri DS0384 strain of the present invention, and may be used to produce more the Lactobacillus reuteri DS0384 strain or its culture broth in a shorter time.

The fermentation starter composition may further comprise a medium in which the Lactobacillus reuteri DS0384 strain can grow. The medium may include, but is not limited to, ingredients such as glucose, yeast extract, proteose peptone, polysorbate 80, ammonium citrate, magnesium sulfate, dipotassium phosphate, and sodium acetate, and may include, without limitation, any ingredient that can help the growth of the Lactobacillus reuteri DS0384 strain. The pH of the medium may be adjusted to be in the range of pH 5 to 7.

The fermentation starter composition may not comprise bacteria other than the Lactobacillus reuteri DS0384 strain. In order to remove bacteria other than the Lactobacillus reuteri DS0384 strain, the fermentation starter composition may be prepared by culturing the Lactobacillus reuteri DS0384 strain in a sterilized culture medium.

The fermentation starter composition may further comprise a cryoprotectant. The cryoprotectant may be one or more selected from the group consisting of, but is not limited to, skim milk powder, maltodextrin, dextrin, trehalose, maltose, lactose, mannitol, cyclodextrin, glycerol and honey, and may include any agent that can prevent damage or death of lactic acid bacteria during a freeze-drying process.

When the fermentation starter composition comprises a cryoprotectant, it may be used in the form of a lyophilisate, for example, a powder, through a freeze-drying process, has advantageous advantages in formulation, packaging, storage, and the like, and may preserve the Lactobacillus reuteri DS0384 strain contained therein for a long time.

### 2. Use of a Composition Comprising a DS0384 Strain or Its Culture Broth for Promoting Intestinal Development, Intestinal Maturation, or Recovery of Intestinal Damage

Another aspect of the present invention provides a composition for promoting intestinal development, intestinal maturation, or recovery of intestinal damage.

The composition comprises a Lactobacillus reuteri DS0384 strain or its culture broth as an active ingredient.

The description of the Lactobacillus reuteri DS0384 strain is the same as described above. The Lactobacillus reuteri DS0384 strain has excellent activity of increasing the size and the like of the intestine and increasing the expression of mature intestinal marker genes, and has excellent activity of increasing the surface area of the damaged intestine to recover it and increasing the expression of intestinal wall function marker and proliferation marker proteins, and thus, the composition comprising the strain or its culture broth as an active ingredient may be usefully used for the purpose of promoting intestinal development, intestinal maturation, or recover of intestinal damage.

The culture broth is obtained by culturing the Lactobacillus reuteri DS0384, and may be the culture broth itself containing bacteria of the strain, or the culture supernatant obtained by removing bacteria therefrom, and may be a filtrate, concentrate, or dry matter thereof. The culture broth from which bacteria were removed contains ingredients, for example, metabolites, produced and secreted by the Lactobacillus reuteri DS0384 strain, and thus may have intestinal development or intestinal maturation activity.

The filtrate is only a water-soluble supernatant except for the precipitate collected by removing the floating solid particles from the culture broth of Lactobacillus reuteri DS0384, and the particles may be filtered using a filter such as cotton or nylon, for example, a filter of 0.2 µm to 5 µm, or cryofiltration, centrifugation, and the like may be used without limitation.

The concentrate is the culture broth in which the concentration of the solid content is increased, and may be a concentrate of the culture broth containing the lactic acid bacteria cells or a concentrate of the culture supernatant from which the lactic acid bacteria cells are removed. The concentrate may be, but is not limited to, those concentrated by vacuum concentration, plate-type concentration, thin film concentration, or the like, for example, it may be carried out at a temperature of 40° C. to 60° C. using a known concentrator. The content of the culture broth included in the composition of the present invention may be appropriately adjusted according to the concentration of the concentrate.

The dry matter includes, but is not limited to, those dried through methods such as freeze drying, vacuum drying, hot air drying, spray drying, reduced pressure drying, spray drying, foam drying, high frequency drying, and infrared drying.

The Lactobacillus reuteri DS0384 strain may be included in the composition at a concentration of 10⁹ to 10¹² CFU/g, and, for example, may be included at a concentration of 10⁹ to 10¹¹ CFU/g, 10¹⁰ to 10¹² CFU/g, or 10¹⁰ to 10¹¹ CFU/g. When the Lactobacillus reuteri DS0384 strain is included in the composition at a concentration within the above range, the Lactobacillus reuteri DS0384 strain may promote intestinal development, intestinal maturation or recovery of intestinal damage, or may sufficiently produce or secrete substances that help them, or may enable normal metabolism without inhibiting the growth of the strain, and thus, the composition of the present invention may exhibit a sufficient effect to be used for the purpose of promoting intestinal development or intestinal maturation.

The composition may further comprise a cryoprotectant. The description of the cryoprotectant is the same as described above. When a lyophilisate obtained by freeze-drying the culture broth containing Lactobacillus reuteri DS0384 cells is used, there is an advantage in oral intake, formulation, packaging, storage, and the like of a composition comprising the same, and there is an advantage in that the freeze-dried strain may show normal physiological activity again and perform growth and metabolism in a subject to which the composition of the present invention is administered, especially in the intestine because the strain may be preserved for a long period of time.

In the present invention, the term “intestine” refers to the region of the digestive tract extending from the stomach to the anus. In humans and other mammal animals, the intestine consists of two regions, the small intestine (further subdivided into duodenum, jejunum, and ileum in humans) and the large intestine (further subdivided into caecum and colon in humans), and other mammal animals may have more complex intestines, and the intestines in the present invention may include all of them.

The intestinal development or maturation may be, specifically, the development or maturation of the small intestine or large intestine. The composition of the present invention may be used for the purpose of promoting the small intestine or large intestine to become more mature, or promoting the maturation of the small intestine or large intestine in an immature state.

In addition, promoting the intestinal development or intestinal maturation is to promote the formation of a specified mature epithelium, or to promote a part of the process of augmenting, increasing, growing, supporting or advancing the differentiation process of enterocytes and intestinal compartments.

The development or maturation of the small intestine may be to increase the length and area of the villus or increase the depth of the crypt in the small intestine.

The development or maturation of the large intestine may be to increase the depth of the crypt or increase the mucosa/submucosa ratio in the large intestine. For example, the development or maturation of the large intestine may be to increase the thickness of the mucosa and not change the thickness of the submucosa. As the development and maturation of the large intestine progresses, the submucosa maintains its structure, and the thickness of the mucosa increases, so that the ratio of mucosa/submucosa constituting the large intestine may increase.

The intestinal development or intestinal maturation may be to increase the expression of one or more genes selected from the group consisting of CDX2, DPP4, OLFM4, DEFA5, CREB3L3, KRT20, LYZ, LCT, SLC5A1, and MUC13.

The intestinal damage may be, for example, caused by an inflammatory response in the intestine, but the cause of the intestinal damage is not limited thereto, and if the function or structure of the intestine is damaged compared to a normal state, the Lactobacillus reuteri strain of the present invention may promote their recovery.

The recovery of intestinal damage may be to restore the intestine to its original state by increasing the surface area of the damaged intestine, or to increase the expression of gene or protein markers related to the function or proliferation of the intestinal wall in the damaged intestine. The intestinal wall function marker may be, but is not limited to, a ZO-1 protein, and the intestinal wall proliferation marker may be, but is not limited to, a Ki67 protein.

The subject for applying the composition of the present invention may be a neonate or infant. In addition, the neonate or infant may be a preterm baby or premature baby. The neonate refers to a baby from immediately after delivery until acquiring the ability to adapt to an independent extrauterine life, and, for example, may be a baby under 28 days of age. The infant may be a baby under 5 years of age, such as under 4 years of age or under 3 years of age. The advantages and effects of the composition of the present invention described above may appear more remarkably when applied to neonates or infants. The preterm baby refers to a baby born earlier than the general gestation period, and, for example, may be a baby born between 29 and 38 weeks of pregnancy. The premature baby refers to a baby born in a state of immature development of the body, and, for example, may be a baby with a low birth weight (for example, 2.5 kg or less, 2 kg or less, or 1.5 kg or less) or a baby with reduced immune capacity. The cause of premature birth and/or immaturity may be a maternal disease, maternal age, stress, fetal condition, and the like, but the composition of the present invention may be applied to the subject without being limited to the above causes. The composition may be applied in a neonatal state immediately after delivery of the preterm baby or premature baby, or may be applied during the period when the preterm baby or premature baby grows after birth and is an infant.

Since the composition of the present invention has the excellent effect of helping intestinal development and maturation, it has the effect capable of improving immature intestinal development of a person to whom it is applied accordingly, and thus, the effect may appear more remarkably when used for the purpose of targeting the preterm baby or premature baby.

The composition comprising the Lactobacillus reuteri DS0384 strain or its culture broth may be prepared in a unit-dose form by formulating the compound of the present invention with carriers, excipients and/or additives according to methods which may be easily carried out by those skilled in the art, or prepared by incorporating it into a multi-dose container. In this case, the formulation may be in the form of a solution, suspension, or emulsion in an oil or aqueous medium, or in the form of an extract, a powder, a granule, a tablet, a capsule, a gel (for example, a hydrogel), or a lyophilisate, and may further comprise a dispersing agent, a stabilizer and/or a cryoprotectant as the additives.

Specifically, the lyophilisate is used in the form of a powder by freeze-drying the strain or its culture broth together with a cryoprotectant, wherein the cryoprotectant may be skim milk powder, maltodextrin, dextrin, trehalose, maltose, lactose, mannitol, cyclodextrin, glycerol and/or honey. In addition, it is used by mixing with a preservative carrier, adsorbing, and then drying, and solidifying, wherein the preservative carrier may be diatomaceous earth, activated carbon, and/or defatted rice bran.

The composition of the present invention may be prepared through the step of mixing the strain or its culture broth with any one of the carriers, excipients or additives.

The description of the strains, carriers, excipients and additives is the same as described above. When the cryoprotectant is used as the additives, the composition comprising the lactic acid bacteria may be prepared in the form of a lyophilized powder by mixing the strain and the cryoprotectant, subjecting the mixture to a freezing process at -45° C. to -30° C., and then drying at 30° C. to 40° C. and grinding with a mixer. Specifically, the freezing process may be a process of vacuum freezing for 65 to 75 hours under a temperature condition of -45° C. to -30° C. and a pressure condition of 5 to 50 mTorr.

### 3. Use of a Composition Comprising a DS0384 Strain or Its Culture Broth for Preventing, Treating and Improving Intestinal Development Disorders or Inflammatory Bowel Disease

Another aspect of the present invention provides a pharmaceutical composition for preventing or treating intestinal development disorders or inflammatory bowel disease, a health functional food composition for preventing or improving intestinal development disorders or inflammatory bowel disease, and a feed composition for preventing or improving intestinal development disorders or inflammatory bowel disease.

The pharmaceutical composition, the health functional food composition and the feed composition comprises a Lactobacillus reuteri DS0384 strain or its culture broth as an active ingredient.

The description of the Lactobacillus reuteri DS0384 strain is the same as described above. The Lactobacillus reuteri DS0384 strain has the excellent activity of increasing the size and the like of the intestine and increasing the expression of mature intestinal marker genes, and has the excellent activity of increasing the surface area of the damaged intestine to recover it and increasing the expression of the intestinal wall function marker and proliferation marker proteins, and thus, the composition comprising the strain or its culture broth as an active ingredient may be usefully used for the purpose of preventing, treating or improving intestinal development disorders. In addition, the Lactobacillus reuteri DS0384 strain increases the surface area of the damaged intestine again and increases the expression of marker proteins related to intestinal wall function and proliferation, and thus, it has the excellent activity of promoting recovery of intestinal damage. Therefore, the composition comprising the strain or its culture broth as an active ingredient may be usefully used for the purpose of preventing, treating or improving inflammatory bowel disease.

The intestinal development disorders may be a state in which the intestinal tract does not function at a normal level due to insufficient development of the intestinal tract when compared to the intestinal tract of humans or non-human animals that have completed development, maturation, differentiation, or growth, and includes intestinal conditions of fetuses, neonates, infants, and the like who are still in the developmental stage or growing stage. In addition, the intestinal development disorders may be a state in which the degree of intestinal development is insufficient compared to the average level of intestinal development based on the same period of the developmental stage or growth stage in which the intestinal development is in progress. The intestinal development disorders include both diseases caused by insuffaicient degree of intestinal development, and related gastrointestinal diseases. Specifically, the intestinal development disorders may be, but are not limited to, a disorder syndrome (malabsorption syndrome), inflammatory bowel disease (Crohn’s disease, ulcerative colitis, and the like), irritable bowel syndrome, short bowel syndrome (SBS), necrotizing enteritis (NEC), radiation proctitis, or preterm babies, premature babies and infants with immature intestines.

The inflammatory bowel disease refers to a disease that occurs as intestinal damage occurs, and specifically may be a state in which an inflammatory response occurs due to damage occurring in the intestine. Specifically, the inflammatory bowel disease may be, but is not limited to, ulcerative colitis, Crohn’s disease, Behcet’s disease, and the like, and may include any disease in which abnormal chronic inflammation occurs in the intestine regardless of the cause of the inflammation. The Lactobacillus reuteri strain of the present invention has the excellent activity of promoting recovery of intestinal damage, such as promoting the proliferation of the intestine inflamed by the injury, and thus, the composition of the present invention may be usefully used for the purpose of preventing, treating or improving inflammatory bowel disease.

In a specific embodiment of the present invention, it was confirmed that excellent effects appeared when human intestinal organoids prepared using human stem cells and human intestinal stem cells isolated therefrom were treated with the Lactobacillus reuteri DS0384 strain, and thus, the composition of the present invention has been experimentally proven to exhibit sufficient effects even when applied to humans, and may be usefully used for the purpose of application to humans.

The pharmaceutical composition of the present invention may be prepared in a unit-dose form by formulating the compound of the present invention with a pharmaceutically acceptable carrier and/or excipient according to methods which may be easily carried out by those skilled in the art, or may be prepared by incorporating them into a multi-dose container. In this case, the formulation may be in the form of a solution, suspension, or emulsion in an oil or aqueous medium, or in the form of an extract, a powder, a granule, a tablet, a capsule, or a gel (for example, a hydrogel), and may further comprise a dispersing agent and/or a stabilizer.

In addition, the strain or its culture broth included in the pharmaceutical composition may be delivered in pharmaceutically acceptable carriers such as colloidal suspensions, powders, saline solutions, lipids, liposomes, microspheres, or nanospherical particles. They may be complexed with or associated with a delivery means, and may be delivered in vivo using delivery systems known in the art, such as lipids, liposomes, microparticles, gold, nanoparticles, polymers, condensation reagents, polysaccharides, polyamino acids, dendrimers, saponins, adsorption enhancing materials or fatty acids.

In addition, the pharmaceutically acceptable carriers may include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, mineral oil, and the like, which are commonly used in the formulations. In addition, a lubricant, a wetting agent, a sweetener, a flavoring agent, an emulsifier, a suspending agent, a preservative, and the like may be further included, in addition to the ingredients as described above. Suitable pharmaceutically acceptable carriers and agents are described in detail in Remington’s Pharmaceutical Sciences, 19th ed., 1995.

The pharmaceutical composition may be administered orally or parenterally upon clinical administration and used in the form of general pharmaceutical formulation. That is, the pharmaceutical composition of the present invention may be administered in a variety of oral and parenteral formulations upon actual clinical administration, and are prepared using diluents or excipients, such as a filler, an extender, a binder, a wetting agent, a disintegrating agent, a surfactant, which are commonly used when formulated. Solid formulations for oral administration include a tablet, a pill, a powder, a granule, a capsule, and the like, and such solid formulations are prepared by mixing at least one or more excipients, for example, starch, calcium carbonate, sucrose or lactose, gelatin, and the like, with the herbal extract or herbal fermented product. In addition to simple excipients, lubricants such as magnesium stearate and talc are also used. Liquid formulations for oral administration include a suspension, a solution for internal use, an emulsion, a syrup, and the like, and may include various excipients, such as a wetting agent, a sweetener, a perfuming agent, a preservative, and the like, in addition to commonly used simple diluents such as water and liquid paraffin. Formulations for parenteral administration include a sterile aqueous solution, a non-aqueous solution, a suspension, an emulsion, a lyophilized formulation, and a suppository. As the non-aqueous solvent and the suspension solvent, propylene glycol, polyethylene glycol, vegetable oils such as olive oil, injectable esters such as ethyl oleate, and the like may be used. As the base of the suppository, witepsol, macrogol, tween 61, cacao butter, laurin, glycerol, gelatin, and the like may be used.

The pharmaceutical composition may be used alone or in combination with surgery, radiotherapy, hormone therapy, chemotherapy, and methods using biological response modifiers, for preventing or improving intestinal development disorders.

The concentration of the active ingredient included in the pharmaceutical composition may be determined in consideration of the purpose of treatment, the patient’s condition, the required period, and the like, and is not limited to a concentration within a specific range. The pharmaceutical composition of the present invention is administered in a pharmaceutically effective amount. In the present invention, the term “pharmaceutically effective amount” refers to an amount sufficient to treat a disease at a reasonable benefit/risk ratio applicable to medical treatment, and the level of the effective dose may be determined depending on the factors including the type and severity of the patient’s disease, the activity of the drug, the sensitivity to the drug, the time of administration, the route of administration and the rate of the excretion, the duration of treatment, and the drugs used simultaneously, and other factors well known in the medical field. The pharmaceutical composition may be administered as an individual therapeutic agent or administered in combination with other therapeutic agents for intestinal developmental disorders, may be administered concurrently, separately, or sequentially with conventional therapeutic agents, and may be administered single or multiple times. Considering all of the above factors, it is important to administer an amount that can achieve the maximum effect in a minimal amount without side effects, which can be easily determined by those skilled in the art.

Specifically, the effective amount of the pharmaceutical composition of the present invention may vary depending on the patient’s age, sex, condition, and body weight, the degree of absorption of the active ingredient into the body, the rate of the inactivation, the rate of the excretion, the type of disease, and concomitant drugs, and may be increased or decreased depending on the route of administration, the severity of obesity, sex, body weight, age, and the like, and, for example, may be administered in an amount of about 0.0001 µg to 500 mg, for example, 0.01 µg to 100 mg per 1 kg of patient’s body weight per day. In addition, it may be administered in a divided dose several times a day, for example, 2 to 3 times a day, at regular time intervals according to the judgment of a medical practitioner or pharmacist.

Another aspect of the present invention provides a method of preventing or treating intestinal developmental disorders, comprising administering the pharmaceutical composition to a subject.

The subject may be a human or a non-human animal, and may be a subject whose degree of development is insufficient compared to the intestinal tract of a human or non-human animal that has completed development, or a subject who is in the developmental stage or growth stage. In addition, the subject may be a human or non-human animal whose degree of intestinal development is insufficient compared to the average level of intestinal development based on the same period of the developmental stage or growth stage in which the intestinal development is in progress.

The description of the formulation of the pharmaceutical composition, the method of administration, the dosage and the concentration of the active ingredient contained in the composition is the same as described above.

The health functional food composition of the present invention may prevent or improve intestinal developmental disorders in humans or non-human animals by promoting intestinal development or intestinal maturation.

When the health functional food composition is used as a food additive, the health functional food composition may be added as it is or used together with other foods or food ingredients, and may be appropriately used according to a conventional method. The amount of the active ingredient may be appropriately used depending on the purpose of its use (prevention or improvement). In general, in the production of food or beverage, the health functional food composition of the present invention is added in an amount of 15 parts by weight or less, preferably 10 parts by weight or less, based on the raw material. However, in the case of long-term ingestion for health purposes, the amount may be less than the above range, and the active ingredient may be used in an amount greater than the above range because there is no problem in terms of safety.

There is no particular limitation on the type of the health functional food. Food to which the health functional food composition can be added may be a probiotic formulation, and, for example, includes meat, sausage, bread, chocolate, candy, snacks, confectionery, pizza, ramen, other noodles, gum, dairy products including ice cream, various soups, beverages, tea drinks, alcoholic beverages, vitamins complexes, fermented foods, and the like, and includes all health foods in a conventional sense. In particular, the fermented food may be, but is not limited to, yogurt (hard type, soft type, drink type), fermented milk such as lactic acid bacteria beverage, cheese, or butter, and any food or product manufactured by fermentation performed by fermenting microorganisms or lactic acid bacteria may also be included.

In addition, the health functional food composition may be prepared as a food, particularly a functional food. The functional food includes ingredients commonly added during food production, and, for example, may include proteins, carbohydrates, fats, nutrients, and seasonings. For example, when manufactured as a drink, natural carbohydrates or flavoring agents may be included as additional ingredients in addition to the active ingredient. The natural carbohydrates are preferably monosaccharides (for example, glucose, fructose, and the like), disaccharides (for example, maltose, sucrose, and the like), oligosaccharides, polysaccharides (for example, dextrin, cyclodextrin, and the like), or sugar alcohols (for example, xylitol, sorbitol, erythritol, and the like). As the flavoring agent, natural flavoring agents (for example, thaumatin, stevia extract, and the like) and synthetic flavoring agents (for example, saccharin, aspartame, and the like) may be used.

In addition to the health functional food composition, various nutrients, vitamins, electrolytes, flavoring agents, coloring agents, pectic acid and its salts, alginic acid and its salts, organic acids, protective colloidal thickeners, pH adjusters, stabilizers, preservatives, glycerin, alcohol, carbonating agents used in carbonated beverages, and the like may be further contained. The ratio of these added ingredients is not very important, but is generally selected from the range of 0.01 to 0.1 parts by weight based on 100 parts by weight of the health functional food composition.

The feed composition of the present invention may prevent or improve intestinal development disorders by promoting intestinal development or intestinal maturation of non-human animals, and may be added as a feed additive composition for the purpose of preventing or improving intestinal development disorders. The feed additive corresponds to an auxiliary feed under the Control of Livestock and Fish Feed Act of Korea.

In the present invention, the term “feed” refers to any natural or artificial diet, one meal, and the like, or ingredients of the one meal, intended for or suitable for eating, ingesting and digesting by animals, the animals are non-human animals. The type of feed is not particularly limited, and feeds commonly used in the art may be used. Non-limiting examples of the feed may include vegetable feeds, such as grains, root fruits, food processing by-products, algae, fibers, pharmaceutical by-products, oils and fats, starches, oil meals or grain by-products; and animal feeds, such as proteins, inorganic materials, oils and fats, minerals, oils and fats, single cell proteins, animal planktons, foods. These feeds may be used alone or in combination of two or more kinds.

Hereinafter, the present invention will be described in detail by way of examples. However, the following examples are only for specifically illustrating the present invention, and the content of the present invention is not limited to the following examples.

All results of the following experiments are expressed as mean ± standard error (s.e.m) for the mean, and all experiments were performed in at least three replicates. P values were determined using a two-tailed t-test, and all analyzes of statistical significance were calculated relative to control groups unless otherwise specified. P values less than 0.05 were considered statistically significant (*P<0.05, **P<0.01, ***P<0.001).

### Example 1. Confirmation of the Effect of the Lactic Acid Bacteria of the Present Invention on Promoting the Maturation of Intestinal Organoids

In order to confirm the effect of the culture broth of the Lactobacillus reuteri DS0384 strain of the present invention on intestinal maturation, intestinal organoids were prepared as an exo vivo intestine model and treated with the culture broth of the DS0384 strain of the present invention, and it was confirmed whether or not the intestinal organoids were matured.

### 1-1. Differentiation of Human Intestinal Organoids

Intestinal organoids were prepared by differentiating human pluripotent stem cells. For the differentiation of human pluripotent stem cells, a method known in the art (Nature 470, 105-109 (2011)) was used. Specifically, for the induction of human pluripotent stem cells (H9 (WA09); WiCell Research Institute, Madison, WI, USA) into definitive endoderm, the stem cells were treated with a medium containing fetal bovine serum (FBS, Thermo Scientific) for 3 days, together with 100 ng/ml of Activin A, wherein the fetal bovine serum was treated while increasing the concentration to 0%, 0.2%, and 2%, respectively. For the formation of hindgut spheroids, they were further cultured for 4 days using a differentiation medium containing 500 ng/ml of FGF4, 3 µm of CHIR 99021 and 2% of fetal bovine serum. The spheroids formed by induction of differentiation were inserted into the Matrigel dome, cultured in a culture medium containing 1X B27 additive (B27 supplement, Invitrogen), 100 ng/ml EGF (R&D Systems), 100 ng/ml Noggin (R&D Systems) and 500 ng/ml R-spondin 1 (R-spondin1, R&D Systems) in a three-dimensional culture environment, subcultured once every 10 days, and were used in experiments.

### 1-2. Confirmation of the Effect of the Lactobacillus reuteri Strain on Promoting the Maturation of Intestinal Organoids

The intestinal organoids differentiated according to Example 1-1 above were treated with several species of lactic acid bacteria to confirm whether or not the intestinal organoids were matured.

The intestinal organoids were treated with a total of 5 types of lactic acid bacteria to confirm their respective effects, morphological changes were confirmed in the intestinal organoids treated with the broth of the lactic acid bacteria, and it was confirmed whether or not intestinal maturation-markers were expressed by immunofluorescence staining (immunocytochemistry, ICC) and qRT. The lactic acid bacteria used were Bifidobacterium longum DS0431 (B. longum DS0431, isolated from neonatal feces), Lactobacillus gasseri DS0444 (L. gasseri DS0444, isolated from breast milk), Lactobacillus curvatus AB70 (L. curvatus AB70, isolated from female genitalia), Lactobacillus rhamnosus DS0979 (L. rhamnosus DS0979, isolated from breast milk), and the Lactobacillus reuteri DS0384 (L. reuteri DS0384, isolated from neonatal feces) strain of the present invention. The culture broth of the five microorganism strains was centrifuged at 12,000 rpm for 10 minutes with a centrifuge, and then, only the supernatant was collected. The collected supernatant was low-temperature sterilized in a heat block preheated to 65° C. for 30 minutes, and then filtered with a 0.22 µm syringe filter unit to remove impurities. They were treated with the culture broth separated in this way, which was diluted 1/100 in the culture medium of the intestinal organoids, and subcultured twice for a total of 20 days, and then, changes in the intestinal organoids were observed.

First, morphological changes of the intestinal organoids were confirmed by checking the size change and the number of budding structures of the intestinal organoids. Specifically, through pictures taken by observing intestinal organoids under a microscope, the size of 6 organoids for each group treated with lactic acid bacteria was measured and compared, and the number of budding structures was confirmed by the number of budding structures generated per organoid for 6 organoids for each group treated with lactic acid bacteria.

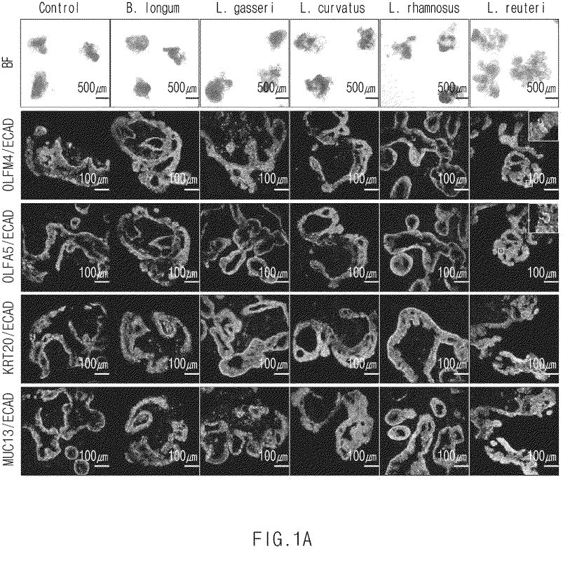

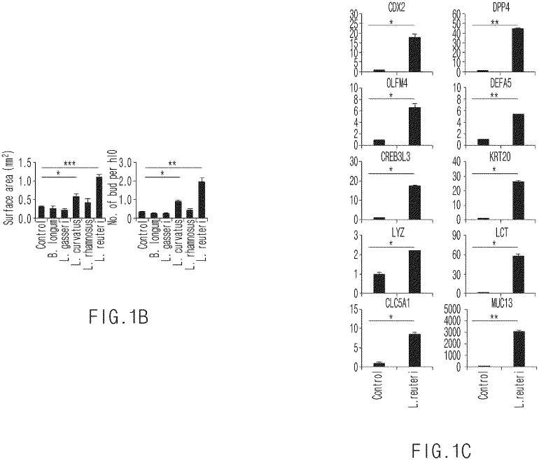

As a result, it could be confirmed that when treated with the culture broth of the Lactobacillus reuteri DS0384 strain of the present invention, larger intestinal organoids were observed and more budding structures were formed, compared to the groups treated with the culture broth of the other four types of lactic acid bacteria (upper data in FIG. 1A, and FIG. 1B). The surface area of the organoids was measured to be 0.30±0.02 mm² for the control group, 0.27±0.06 mm² for the group treated with the culture broth of B. longum strain, 0.24±0.05 mm² for the group treated with the culture broth of L. gasseri, 0.56±0.08 mm² for the group treated with the culture broth of L. curvatus, 0.41±0.10 mm² for the group treated with the culture broth of L. rhamnosus, and 1.10±0.07 mm² for the group treated with the culture broth of the L. reuteri (DS0384) strain of the present invention, and thus, the size of organoids significantly increased in the groups treated with the culture broth of L. curvatus and the L. reuteri (DS0384) strain of the present invention, and among them, the surface area of organoids increased the most in the group treated with the culture broth of the L. reuteri (DS0384) strain (left panel in FIG. 1B). As a result of measuring the number of budding structures, the number of budding structures formed was measured to be 3.17±0.52 for the control group, 2.33±0.61 for B. longum, 2.50±0.37 for L. gasseri, 8.83±0.96 for L. curvatus, 4.33±0.78 for L. rhamnosus, and 19.67±2.20 for the L. reuteri (DS0384) strain of the present invention. It was confirmed that the number of budding structures significantly increased in the groups treated with the culture broth of L. curvatus and the L. reuteri (DS0384) strain of the present invention, and among them, increased the most in the group treated with the culture broth of the L. reuteri (DS0384) strain (right panel in FIG. 1B) panel).

In addition, the expression level of the mature intestinal marker proteins to be expressed in the mature intestine was confirmed by immunofluorescence staining. As the marker proteins, OLFM4, a marker for mature intestinal stem cells, DEFA5, a marker for mature paneth cells, KRT20, a marker for a mature intestinal structural protein, and MUC13, a marker for mucin-producing cells were targeted. Specifically, the intestinal organoids treated with the culture broth of the five lactic acid bacteria as described above were fixed in 4% PFA (paraformaldehyde), and then cryoprotected by treatment with a 10-30% sucrose solution, and frozen by treatment with an OCT solution. Frozen intestinal organoid tissue was cut to a thickness of 10-20 µm with a microtome to make a section, and treated with PBS containing 0.1% Triton X-100 to permeate the section, and then blocked with PBS containing 4% BSA (bovine serum albumin) for 1 hour. It was reacted overnight at 4° C. using primary antibodies against the above four target marker proteins, anti-OLFM4 antibody (ab85046, abcam, Cambridge, MA, USA), anti-DEFA5 antibody (ab90802, abcam), anti-KRT20 antibody (ab76126, abcam) and anti-MUC13 antibody (ab124654, abcam), which were diluted 1:100, respectively, and then reacted at room temperature for 1 hour using secondary antibodies, anti-goat antibody (anti-goat IgG Alexa Fluor 488, A21467, Invitrogen), anti-rabbit antibody (anti-rabbit IgG Alexa Fluor 594, A21442, Invitrogen), anti-mouse antibody (anti-mouse IgG Alexa Fluor 594, A21203, Invitrogen), which were diluted 1:200, respectively, and then, the nucleus was stained with DAPI and observed under a fluorescence microscope.

As a result, it could be confirmed that OFLM4, DEFA5, KRT20 and MUC13 proteins were stained and observed only in the group treated with the culture broth of the Lactobacillus reuteri DS0384 strain of the present invention, and thus, proteins that appeared during intestinal maturation were expressed and produced (lower data in FIG. 1A), whereas the expression of proteins related to intestinal maturation was not observed in the groups treated with the culture broth of the other 4 types of lactic acid bacteria.

Furthermore, the expression levels of mature intestinal marker genes in intestinal organoids treated with the culture broth of each lactic acid bacteria were confirmed through qRT-PCR. As the mature intestinal marker genes, CDX2, DPP4, OLFM4, DEFA5, CREB3L3, KRT20, LYZ, LCT, SLC5A1 and MIC13 genes were targeted. RNA was isolated and extracted from the intestinal organoids treated with the culture broth using the RNeasy kit (Qiagen) according to the manufacturer’s protocol, and cDNA was synthesized by reverse transcription of mRNA using a kit for cDNA synthesis (Superscript IV cDNA synthesis system, Invitrogen). qRT-PCR was performed on the synthesized cDNA with a PCR machine (7500 Fast Real-time PCR system, Invitrogen) using the primers according to Table 1 below.

As a result, when treated with the culture broth of the Lactobacillus reuteri DS0384 strain of the present invention, the expression level of all 10 intestinal maturation marker genes including the CDX2 gene increased several to several thousand times compared to the control group, and thus, a significant increase could be confirmed (FIG. 1C).

TABLE 1

| SEQ ID NO: | Sequence name | Sequence (5′-> 3′) |
|---|---|---|
| 1 | GAPDH forward primer | GAAGGTGAAGGTCGGAGTC |
| 2 | GAPDH reverse primer | GAAGATGGTGATGGGATTTC |
| 3 | CDX2 forward primer | CTGGAGCTGGAGAAGGAGTTTC |
| 4 | CDX2 reverse primer | ATTTTAACCTGCCTCTCAGAGAGC |
| 5 | DPP4 forward primer | CAAATTGAAGCAGCCAGACA |
| 6 | DPP4 reverse primer | GGAGTTGGGAGACCCATGTA |
| 7 | OLFM4 forward primer | ACCTTTCCCGTGGACAGAGT |
| 8 | OLFM4 reverse primer | TGGACATATTCCCTCACTTTGGA |
| 9 | DEFA5 forward primer | CCTTTGCAGGAAATGGACTC |
| 10 | DEFA5 reverse primer | GGACTCACGGGTAGCACAAC |
| 11 | CREB3L3 forward primer | ATCTCCTGTTTGACCGGCAG |
| 12 | CREB3L3 reverse primer | GTCGTCAGAGTCGGGGTTTG |
| 13 | KRT20 forward primer | TGGCCTACACAAGCATCTGG |
| 14 | KRT20 reverse primer | TAACTGGCTGCTGTAACGGG |
| 15 | LYZ forward primer | AAAACCCCAGGAGCAGTTAAT |
| 16 | LYZ reverse primer | CAACCCTCTTTGCACAAGCT |
| 17 | LCT forward primer | GGCAGTCTGGGAGTTTTAGG |
| 18 | LCT reverse primer | ATGCCAAAATGAGGCAAGTC |
| 19 | SLC5A1 forward primer | GTGCAGTCAGCACAAAGTGG |
| 20 | SLC5A1 reverse primer | ATGCACATCCGGAATGGGTT |
| 21 | MUC13 forward primer | CGGATGACTGCCTCAATGGT |
| 22 | MUC13 reverse primer | AAAGACGCTCCCTTCTGCTC |

Considering the results of the above experiments together, when human intestinal organoids were treated with the Lactobacillus reuteri DS0384 strain of the present invention among various types of lactic acid bacteria isolated from neonatal feces, breast milk or female genitalia, it was confirmed that the expression level of genes and proteins related to intestinal maturation significantly increased, and it can be seen that it has the effect of actually promoting intestinal development and maturation of the intestine, such as increasing the size of intestinal organoids and inducing the formation of budding structures.

### 1-3. Confirmation of the Difference in the Effect of Promoting the Maturation of Intestinal Organoids Between L. reuteri Strains

Through Example 1-2 above, it was confirmed that the Lactobacillus reuteri DS0384 strain of the present invention had the excellent effect of promoting intestinal maturation and development compared to the lactic acid bacteria belonging to other species. Accordingly, the DS0384 strain of the present invention was compared with other strains classified as the same species to compare the difference in the effect of promoting the maturation of intestinal organoids.

For comparison with the Lactobacillus reuteri DS0384 strain of the present invention, intestinal organoids were treated with the culture broth of the DS0191, DS0195, DS0333 and DS0354 strains among the microorganisms equally classified as Lactobacillus reuteri, and their effects were compared to that treated with the culture broth of the DS0384 strain. The experiment was conducted using the same method as in Example 1-2 above, and morphological changes of intestinal organoids after treatment with the culture broth were confirmed, and the expression level of proteins and genes used as mature intestinal markers was confirmed by immunofluorescence staining and qRT-PCR.

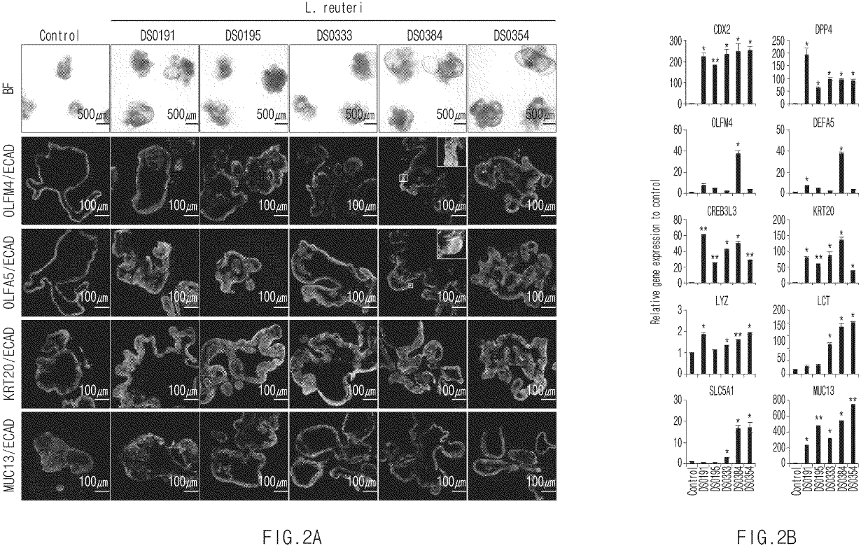

As a result, it could be confirmed that other Lactobacillus reuteri strains also had the effect of promoting the development of intestinal organoids to some extent, but the structure of intestinal organoids was most developed when treated with the culture broth of the DS0384 strain of the present invention (upper data in FIG. 2A), and even in the results of immunofluorescence staining, it could be confirmed that OLFM4 and DEFA5 proteins were expressed only in the group treated with the culture broth of the DS0384 strain of the present invention (lower data in FIG. 2A). Even in the result of confirming the expression level of the marker genes confirmed through qRT-PCR, unlike the fact that the expression of all 8 genes was significantly increased in the group treated with the culture broth of the DS0384 strain of the present invention compared to the control group, the expression of some genes was not increased in the groups treated with the culture broth of other Lactobacillus reuteri strains (FIG. 2b). In particular, it was confirmed that the expression level of OLFM4 gene and DEFA5 gene was remarkably increased in the group treated with the culture broth of the DS0384 strain, unlike the groups treated with the culture broth of other Lactobacillus reuteri strains. The OLFM4 gene is expressed at a high level in the human small intestine and large intestine, and corresponds to a marker gene particularly expressed in intestinal stem cells, and thus, it is known to be closely related to intestinal development and differentiation. The DEFA5 gene is a gene encoding the DEFA5 (defensin alpha 5) protein abundantly present on the surface of the intestine, and is expressed at a high level in mature paneth cells, and thus, it is used as a marker thereof. Therefore, it could be confirmed that the DS0384 strain of the present invention, which exhibits a characteristic of increasing the expression level of OLFM4 gene and DEFA5 gene unlike the groups treated with the culture broth of other microbial strains, may promote intestinal maturation and development even in a different way from other strains, and thus, the maturation promoting effect appeared more remarkably.

In addition to the results of comparative experiments with the 4 types of Lactobacillus reuteri strains confirmed above, the effect of the DS0384 strain of the present invention was reverified through additional comparative experiments with Lactobacillus reuteri DSP007, DS0337 and KCTC3594 strains. In the same manner, intestinal organoids were treated, and the morphological changes and expression levels of marker proteins and genes were confirmed by immunofluorescence staining and qRT-PCR.

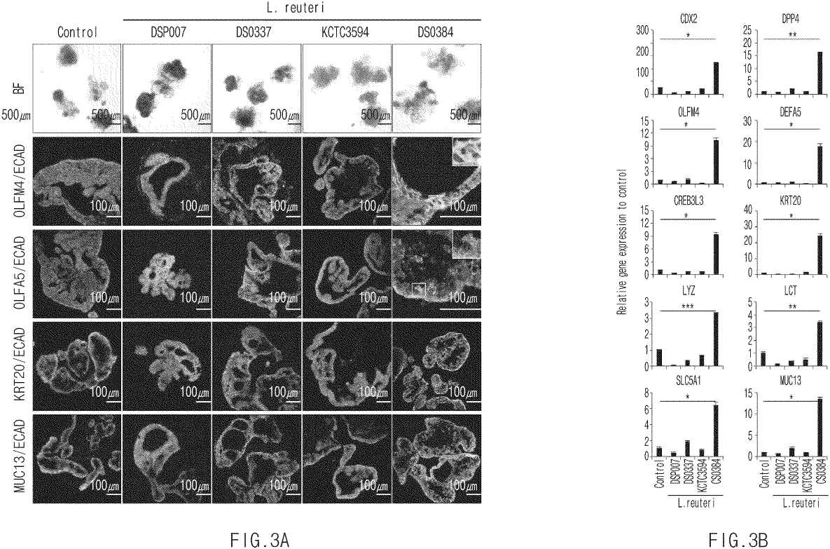

As a result, unlike the fact that the morphological maturation of intestinal organoids was less in the groups treated with the culture broth of Lactobacillus reuteri DSP007, DS0337 and KCTC3594, the maturation of intestinal organoids was conspicuously confirmed in the group treated with the culture broth of the DS0384 strain of the present invention, and in the results of immunofluorescence staining, the expression of OLFM4, DEFA5, KRT20 and MUC13 proteins was observed only in the group treated with the culture broth of the DS0384 strain of the present invention (FIG. 3A). Even in the qRT-PCR result, it could be confirmed that the expression of 10 mature intestinal marker genes was remarkably increased in the group treated with the culture broth of the DS0384 strain of the present invention compared to the control group, whereas the expression level in the groups treated with the culture broth of the other three Lactobacillus reuteri strains appeared remarkably less than that of the culture broth of the DS0384 strain of the present invention and rather appeared less than the control group (FIG. 3B).

Considering the results of Examples 1-2 and 1-3 above together, it can be seen that the culture broth of the Lactobacillus reuteri DS0384 strain of the present invention has the excellent effect of promoting the maturation and development of intestinal organoids. In particular, It was confirmed that lactic acid bacteria strains classified as a species different from Lactobacillus reuteri did not show an effect of promoting intestinal maturation, and the DS0384 strain of the present invention showed a remarkably excellent effect of promoting intestinal maturation compared to the groups treated with the culture broth of other strains belonging to Lactobacillus reuteri.

### 1-4. Comparison of the Effects of Lactobacillus reuteri DS0384 and DSP007 Strains on Promoting the Maturation of Intestinal Organoids, and Optimization of Culture Conditions

Through the experimental results according to Example 1-3 above, an additional comparative experiment was performed with the Lactobacillus reuteri DSP007 strain, which showed an effect of promoting the maturation of intestinal organoids next to the Lactobacillus reuteri DS0384 strain of the present invention, and this case, the culture broth of each strain was obtained and treated for each culture time to confirm the difference in effect according to the culture conditions.

Specifically, while the Lactobacillus reuteri DS0384 strain of the present invention and DSP007 strain were cultured for 6 hours, 12 hours, 18 hours, and 24 hours, intestinal organoids were treated with the culture broth obtained for each time, and the expression level of 10 intestinal maturation marker genes including CDX2 gene along with morphological changes of the intestinal organoids were measured by qRT-PCR in the same manner as in Example 1-2 above.

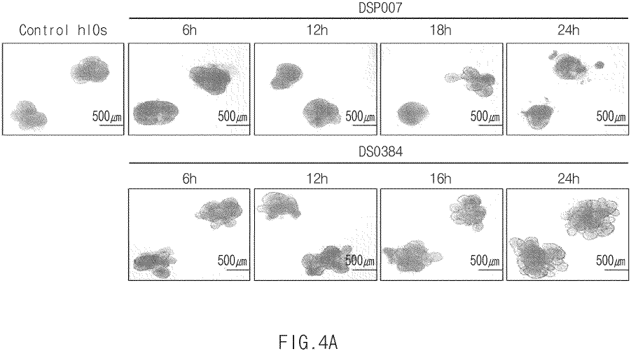

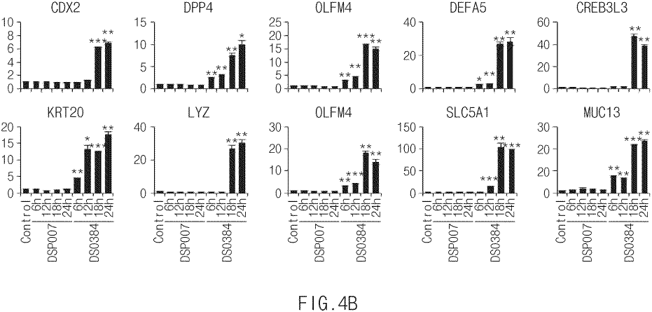

As a result, it could be confirmed that when treated with the culture broth of the Lactobacillus reuteri DS0384 strain, not only the morphological changes of intestinal organoids were clearly observed, but also the expression level of the 10 intestinal maturation marker genes was remarkably increased in the group treated with the culture broth of the DS0384 strain (FIG. 4A and FIG. 4B). In particular, the expression level of the mature intestinal marker genes was significantly increased in the intestinal organoids treated with the culture broth obtained by culturing the DS0384 strain for 18 hours or more (18 hours, 24 hours culture). In contrast, when treated with the culture broth of the DSP007 strain, which is a strain belonging to the same Lactobacillus reuteri, the effect of promoting intestinal maturation was not significantly observed, and no difference in the effect according to the culture time was also confirmed.

From the above experimental results, it could be confirmed once again that the Lactobacillus reuteri DS0384 strain of the present invention or its culture broth was a strain having the excellent effect of promoting intestinal maturation compmared to other Lactobacillus reuteri strains, and it could be also confirmed that the effect of promoting intestinal maturation was more excellent in the culture broth obtained after culturing the DS0384 strain for 18 hours or more, for example, 18 hours or 24 hours.

### 1-5. Confirmation of the Effect of the Lactobacillus reuteri DS0384 Strain on Promoting the Growth of Human Intestinal Stem Cells

In addition to confirming that the Lactobacillus reuteri DS0384 strain of the present invention is excellent in the effect of promoting the growth of intestinal organoids, it was confirmed whether or not the effect of promoting the growth of stem cells appeared when human intestinal stem cells isolated from intestinal organoids were treated with the DS0384 strain.

Specifically, the intestinal stem cells were isolated from the human intestinal organoids, cultured, and attached to a culture dish, and then, the intestinal stem cells were treated with the culture broth of the Lactobacillus reuteri DS0384 strain and the Lactobacillus reuteri KCTC3594 strain diluted in a cell culture medium, and the intestinal stem cells were cultured and observed for 7 to 10 days while replacing the medium every 2 days. In addition, a difference in growth according to the treatment of the culture broth was confirmed by comparing the colony morphology (surface area) of the intestinal stem cells.

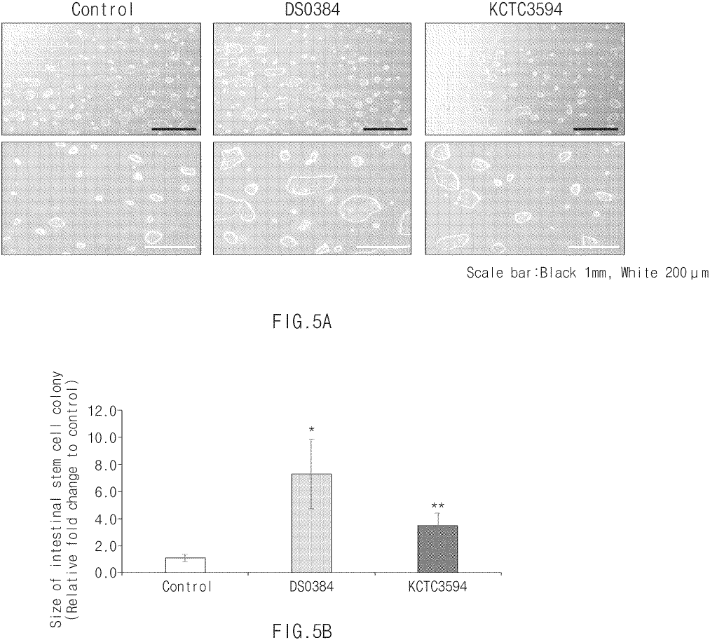

As a result, the surface area of the intestinal stem cell colonies treated with the culture broth of the Lactobacillus reuteri DS0384 strain of the present invention was measured to be increased by about 7.3 times compared to the surface area of the intestinal stem cell colonies treated with the culture broth of the Lactobacillus reuteri KCTC3594 (FIG. 5A and FIG. 5B). Therefore, it could be confirmed that the Lactobacillus reuteri DS0384 strain of the present invention has excellent characteristics not only in the effect of promoting intestinal maturation, but also in the effect of promoting the growth of intestinal stem cells.

### Example 2. Confirmation of the Effect of the Lactic Acid Bacteria of the Present Invention on Protecting Intestinal Organoids

In order to confirm the effect of the culture broth of the Lactobacillus reuteri DS0384 strain of the present invention on protecting the intestine, it was confirmed whether or not the effect of protecting the intestinal organoids appeared, after preparing intestinal organoids, and then inducing their damage and treating them with the culture broth of the DS0384 strain of the present invention, in the same manner as used to confirm the effect of promoting intestinal maturation and the effect of promoting the growth of intestinal stem cells.

First, in order to obtain the damaged intestinal organoids, intestinal organoids of passage 2 or 3 were treated with the inflammatory cytokines IFNγ and TNFα at a concentration of 125 ng/ml, respectively, for 3 days after 10 days of passage to obtain damaged intestinal organoids. In addition, the intestinal organoids were simultaneously treated with the inflammatory cytokines and the culture broth of the Lactobacillus reuteri DS0384 strain of the present invention diluted 1/100 in the culture medium of the intestinal organoids for 3 days, and then, the state of the intestinal organoids was observed.

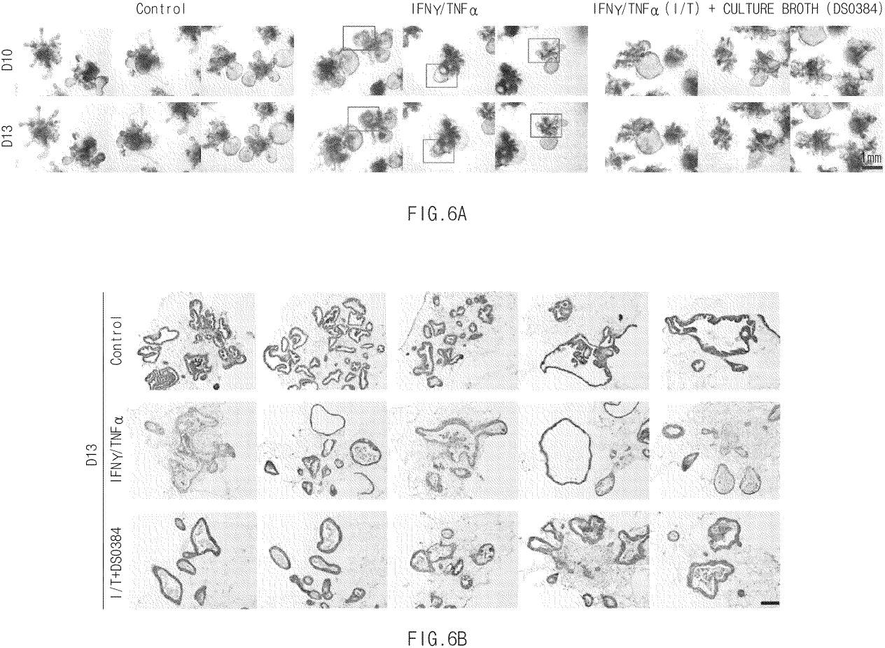

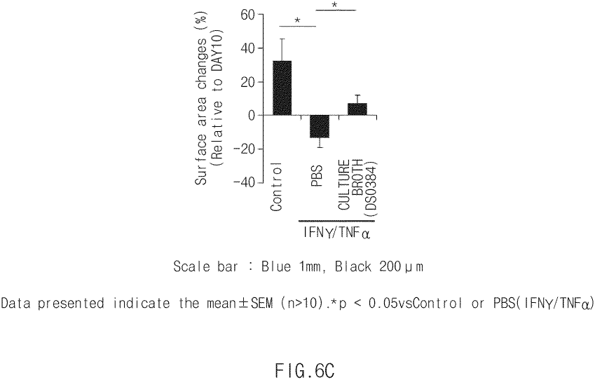

As a result of performing morphological analysis as the damaged intestinal organoids were treated with the culture broth of the DS0384 strain, it could be confirmed that the surface area of the intestinal organoids treated only with inflammatory cytokines decreased, whereas the surface area of the intestinal organoids treated with the culture broth of the DS0384 strain of the present invention was significantly increased and recovered (FIG. 6A, FIG. 6B and FIG. 6C).

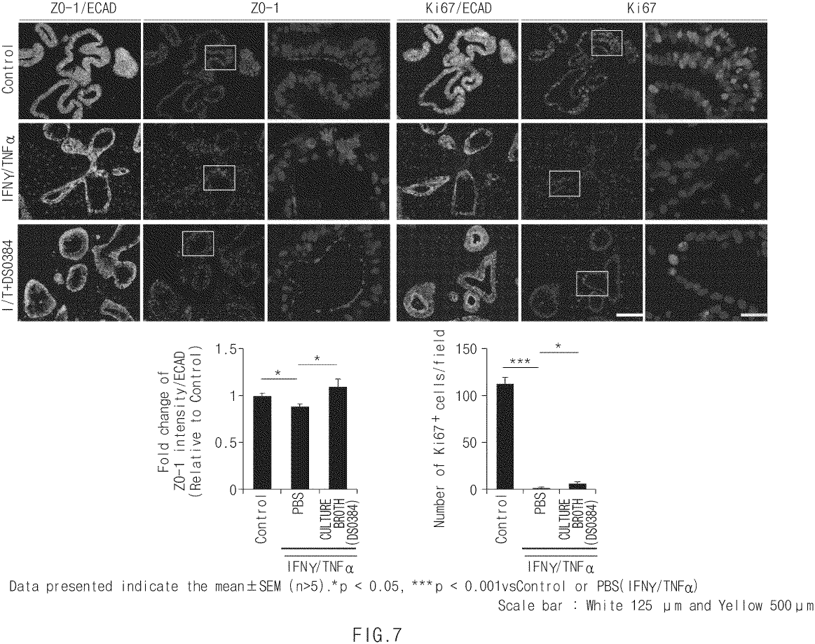

In addition, after cryosectioning the intestinal organoids, the expression of intestinal wall function marker proteins and proliferation marker proteins was confirmed by immunofluorescence staining. As a result, it could be confirmed that the intestinal organoids treated with the culture broth of the DS0384 strain of the present invention significantly increased the expression level of ZO-1, an intestinal wall function marker protein, and Ki67, a proliferation marker protein compared to the intestinal organoids of the control group treated only with inflammatory cytokines (FIG. 7).

Considering the above experimental results together, when the intestinal organoids damaged by treatment with inflammatory cytokines is treated with the culture broth of the Lactobacillus reuteri DS0384 strain of the present invention, it could be confirmed that it has the effect of promoting recovery by inhibiting the decrease in surface area, and exhibits the effect of protecting the intestines against damage by increasing the expression of intestinal wall or proliferation marker proteins. Therefore, it could be confirmed that the Lactobacillus reuteri DS0384 strain of the present invention has an effect of protecting the intestines in addition to the effect of promoting intestinal maturation and growth of intestinal stem cells.

### Example 3. Confirmation of the Effect of the Lactic Acid Bacteria of the Present Invention on Promoting Intestinal Maturation and Development of Animals Using Mouse Experiments

In the exo vivo intestinal model, intestinal organoids and intestinal stem cells made by mimicking the human intestine through Example 1, it was confirmed that the DS0384 strain of the present invention was superior to microorganisms such as other types of Lactobacillus reuteri in the effect of maturing and developing the intestine. Accordingly, in order to confirm whether or not the effect of promoting intestinal maturation appears even when the DS0384 strain of the present invention is actually applied to animals, an animal experiment was performed on mice.

### 3-1. Production of Mouse Model and Design of Animal Experiment

As the mice to be used for the experiments, 3-day old male C57BL/6J mice (DBL, Eumseong, Korea) were used, and all animal experiments were performed with approval from the Institutional Animal Care and Use Committee (IACUC) of KRIBB (Approval No: KRIBB-AEC-19222). The control groups were the group in which the mice were orally gavaged with physiological saline (PBS), and the group in which the mice were orally gavaged with a culture broth of Lactobacillus reuteri KCTC3594, and in the case of the group administered with the DS0384 strain of the present invention, the experiments were performed by administering the culture broth containing bacteria to the mice, or orally gavaging the culture broth from which bacteria were removed by centrifugation in the same manner as in Example 1-2 above.

The 3-day old mice were stabilized for 2 days, and then the mice were orally gavaged with each sample from the experimental groups and the control groups for 7 days, and after 7 days, the mice were humanely euthanized, and the intestines were separated, and changes in length and weight were measured at the time of separation. If growth retardation occurred compared to other mice at the time of oral gavage, it was excluded from the experiment, and 7 or more sets of repeated experiments for each condition were configured to secure statistical significance (n > 7 per group).

### 3-2. Confirmation of the Effect of the DS0384 Strain Of the Present Invention on Promoting Intestinal Development in Mice

The mice of Example 2-1 above were orally gavaged with the culture broth containing the Lactobacillus reuteri DS0384 bacteria of the present invention and the culture broth in which the bacteria were removed by centrifugation for 7 days, respectively, as the experimental groups, and orally gavaged with PBS and the culture broth of Lactobacillus reuteri KCTC3594 for 7 days, respectively, as the control groups, and then, tissue specimens were prepared for microscopic observation. First, a median incision was made in the abdomen of the euthanized mice, and the entire digestive tract was excised. Samples were collected by dividing the isolated digestive tract tissue into the jejunum part of the small intestine and the distal colon part of the large intestine, fixed using 4% PFA, and then cryoprotected with sucrose, and frozen using an OCT solution. Next, 10 µm-thick cryostat sections were prepared using a Cryostat Microtome at -20° C., and for samples that are difficult to observe with cryostat sections, they were fixed with 4% PFA, and then embedded in paraffin, sectioned with a microtome to a thickness of 5-7 µm, and used.

H&E staining (Hematoxylin and eosin staining) was performed to observe the properties of the cryostat sectioned tissue. The cryostat sectioned tissue was adhered to a glass slide, stained with hematoxylin for 5 minutes, washed with running water, and then stained with eosin. Thereafter, it was dehydrated using ethanol, and then washed with xylene, and sealed. The tissue sectioned from the paraffin-embedded tissue with a microtome was adhered to a slide glass, dried, stained with hematoxylin and eosin, and then sealed through the same procedure as the cryostat sectioned tissue.

By observing the histological morphology of the tissue under a microscope, the length and area of the villus and the depth of the crypt were measured for the jejunum of the small intestine, and the depth of the crypt and the thickness of the mucosa were measured for the large intestine, and by analyzing them, the degree of intestinal development and maturation was quantified. As the large intestine matures, the thickness of the mucosa of the large intestine increases, and the structure of the submucosa is maintained during normal development, and thus, the mucosa/submucosa ratio increases. Therefore, the maturity of the large intestine may be confirmed by measuring whether or not the crypt depth and the mucosa/submucosa ratio in the large intestine increase.

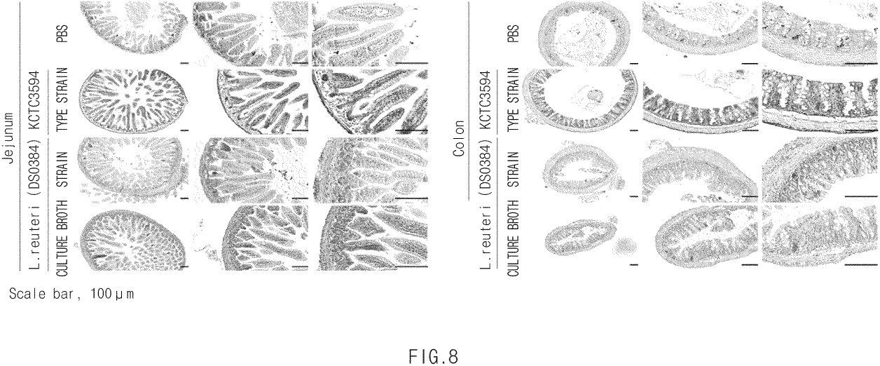

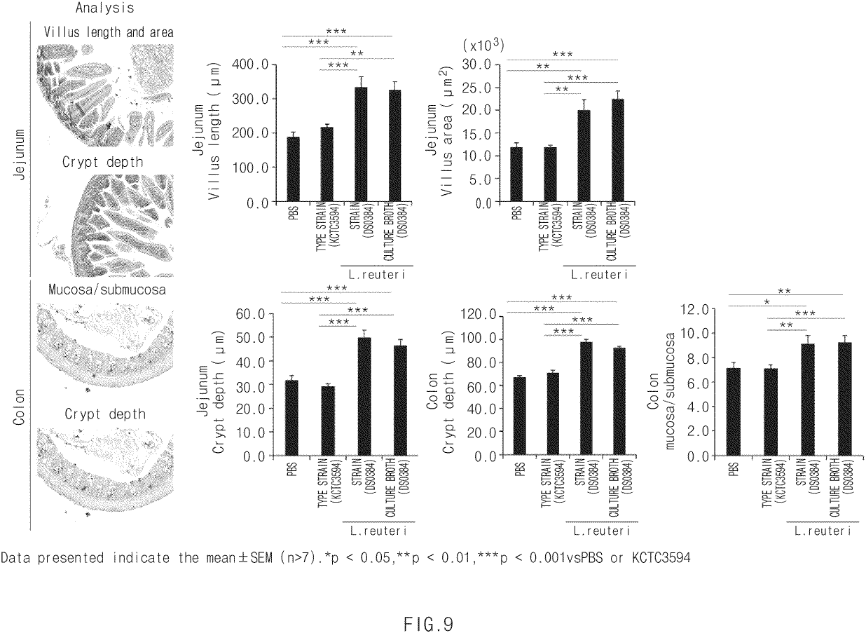

As a result, it was observed that when orally gavaged with the culture broth of the Lactobacillus reuteri DS0384 strain of the present invention, the length and area of the villus increased and the depth of the crypt increased in the small intestine of mice, and the depth of the crypt in the large intestine increased. Specifically, it was confirmed that the development of the small intestine and large intestine was significantly increased when administering the culture broth of DS0384, even when compared to the groups in which PBS and the culture broth of KCTC3594 were administered used as the control groups (FIGS. 8 and 9). Although the KCTC3594 strain is also a microorganism classified as Lactobacillus reuteri like the DS0384 strain, it could be confirmed that when orally gavaged with the culture broth of KCTC3594, no change was shown in terms of development of the small intestine and large intestine compared to the case where PBS was administered, but the culture broth of DS0384 of the present invention promoted significant intestinal development both in the case of administration after including bacteria and in the case of administration after removing bacteria by centrifugation. As a result of treatment with the Lactobacillus reuteri DS0384 strain of the present invention and its culture, it could be confirmed that the length and area of the villus and the crypt length in the small intestine were increased, and thus, the effect of promoting the development of the small intestine was excellent, and it could be confirmed that the mucosa/submucosa ratio was increased, and thus, the effect of promoting the development of the large intestine was excellent.

From the above results, it can be seen that the Lactobacillus reuteri DS0384 strain of the present invention and metabolites produced therefrom have an effect of promoting the development of the small intestine and large intestine, and it can be seen that other Lactobacillus reuteri strains do not have the above effect, or the effect is lower than that of the DS0384 strain. Therefore, the Lactobacillus reuteri DS0384 strain of the present invention and its culture broth are not only suitable for use for the purpose of promoting intestinal maturation, but also are characterized by having a far superior effect of promoting intestinal maturation compared to other conventional lactic acid bacteria and Lactobacillus reuteri strains, which are known in the art and used for various purposes, and thus, it can be seen that the DS0384 strain of the present invention is a novel strain having new effects and characteristics that cannot be predicted or achieved from conventional Lactobacillus reuteri.

In the above, it will be apparent to those skilled in the art that the present invention has been described in detail only for the described embodiments, but various changes and modifications can be made within the scope of technical idea of the present invention, and it is obvious that such changes and modifications fall within the scope of the appended claims.

### ACCESSION NUMBER

Name of depository institution: Korean Collection for Type Cultures, Korea Research Institute of Bioscience and Biotechnology

Accession number: KCTC 14164BP

Accession date: 20200406

1-15. (canceled)

16. A composition comprising Lactobacillus reuteri DS0384 strain deposited under Accession No. KCTC 14164BP or a culture broth thereof.

17. The composition according to claim 16, wherein the composition further comprises a cryoprotectant.

18. A fermentation starter composition comprising a Lactobacillus reuteri DS0384 strain.

19. The fermentation starter composition according to claim 18, wherein the composition further comprises a cryoprotectant.

20. A method of promoting intestinal development, intestinal maturation or recovery of intestinal damage in a subject in need thereof comprising administering an effective amount of the composition according to claim 16 to the subject.

21. The method according to claim 20, wherein the Lactobacillus reuteri DS0384 strain is contained in the composition at a concentration of 10⁹ to 10¹² CFU/g.

22. The method according to claim 20, wherein the composition further comprises a cryoprotectant.

23. The method according to claim 20, wherein the intestinal development or intestinal maturation is the development or maturation of a small intestine or large intestine.

24. The method according to claim 23, wherein the development or maturation of the small intestine is to increase the length and area of the villus or to increase the length of the crypt in the small intestine.

25. The method according to claim 23, wherein the development or maturation of the large intestine is to increase the length of the crypt in the large intestine or to increase the mucosa/submucosa ratio in the large intestine.

26. The method according to claim 20, wherein the intestinal development or intestinal maturation is to increase the expression of one or more genes selected from the group consisting of CDX2, DPP4, OLFM4, DEFA5, CREB3L3, KRT20, LYZ, LCT, SLC5A1, and MUC13.

27. The method according to claim 20, wherein the recovery of intestinal damage is to increase the surface area of the damaged intestine, or to increase the expression of at least one protein selected from the group consisting of ZO-1 and Ki67 proteins in the intestine.

28. A method of preventing or treating intestinal development disorders or inflammatory bowel disease in a subject in need thereof comprising administering an effective amount of the composition according to claim 16 to the subject.

29. The method according to claim 28, wherein the composition is prepared into any one formulation selected from the group consisting of capsules, powders, granules, tablets, pills, and lyophilisates.


SEQUENCE LISTING (22 sequences)

SEQ ID NO: 1 - DNA, 19 bp, Artificial Sequence
  GAPDH forward primer
gaaggtgaaggtcggagtc

SEQ ID NO: 2 - DNA, 20 bp, Artificial Sequence
  GAPDH reverse primer
gaagatggtgatgggatttc

SEQ ID NO: 3 - DNA, 22 bp, Artificial Sequence
  CDX2 forward primer
ctggagctggagaaggagtttc

SEQ ID NO: 4 - DNA, 24 bp, Artificial Sequence
  CDX2 reverse primer
attttaacctgcctctcagagagc

SEQ ID NO: 5 - DNA, 20 bp, Artificial Sequence
  DPP4 forward primer
caaattgaagcagccagaca

SEQ ID NO: 6 - DNA, 20 bp, Artificial Sequence
  DPP4 reverse primer
ggagttgggagacccatgta

SEQ ID NO: 7 - DNA, 20 bp, Artificial Sequence
  OLFM4 forward primer
acctttcccgtggacagagt

SEQ ID NO: 8 - DNA, 23 bp, Artificial Sequence
  OLFM4 reverse primer
tggacatattccctcactttgga

SEQ ID NO: 9 - DNA, 20 bp, Artificial Sequence
  DEFA5 forward primer
cctttgcaggaaatggactc

SEQ ID NO: 10 - DNA, 20 bp, Artificial Sequence
  DEFA5 reverse primer
ggactcacgggtagcacaac

SEQ ID NO: 11 - DNA, 20 bp, Artificial Sequence
  CREB3L3 forward primer
atctcctgtttgaccggcag

SEQ ID NO: 12 - DNA, 20 bp, Artificial Sequence
  CREB3L3 reverse primer
gtcgtcagagtcggggtttg

SEQ ID NO: 13 - DNA, 20 bp, Artificial Sequence
  KRT20 forward primer
tggcctacacaagcatctgg

SEQ ID NO: 14 - DNA, 20 bp, Artificial Sequence
  KRT20 reverse primer
taactggctgctgtaacggg

SEQ ID NO: 15 - DNA, 21 bp, Artificial Sequence
  LYZ forward primer
aaaaccccaggagcagttaat

SEQ ID NO: 16 - DNA, 20 bp, Artificial Sequence
  LYZ reverse primer
caaccctctttgcacaagct

SEQ ID NO: 17 - DNA, 20 bp, Artificial Sequence
  LCT forward primer
ggcagtctgggagttttagg

SEQ ID NO: 18 - DNA, 20 bp, Artificial Sequence
  LCT reverse primer
atgccaaaatgaggcaagtc

SEQ ID NO: 19 - DNA, 20 bp, Artificial Sequence
  SLC5A1 forward primer
gtgcagtcagcacaaagtgg

SEQ ID NO: 20 - DNA, 20 bp, Artificial Sequence
  SLC5A1 reverse primer
atgcacatccggaatgggtt

SEQ ID NO: 21 - DNA, 20 bp, Artificial Sequence
  MUC13 forward primer
cggatgactgcctcaatggt

SEQ ID NO: 22 - DNA, 20 bp, Artificial Sequence
  MUC13 reverse primer
aaagacgctcccttctgctc